(19) United States
(12) Patent Application Publication
(10) Pub. No.: US 2022/0010249 A1
(43) Pub. Date: Jan. 13, 2022

(54) DEVICE FOR PRODUCING MICROALGAE

(71) Applicants: INRIA INSTITUT NATIONAL DE RECHERCHE EN INFORMATIQUE ET EN AUTOMATIQUE, Le Chesnay (FR); INALVE, Nice (FR)

(72) Inventors: Olivier BERNARD, Le Chesnay (FR); Hubert BONNEFOND, Nice (FR)

(21) Appl. No.: 17/294,300

(22) PCT Filed: Nov. 14, 2019
(86) PCT No.: PCT/EP2019/081386

§ 371 Date: May 14, 2021

(30) Foreign Application Priority Data
Nov. 16, 2018 (FR) 1860614

(51) Int. Cl. C12M 1/00; C12M 1/12; C12M 1/26

(52) U.S. Cl. CPC C12M 21/02; C12M 25/02; C12M 41/06; C12M 33/20; C12M 31/10

(57) ABSTRACT

The invention relates to a device (10) for producing microalgae, comprising a basin (12) containing an aqueous medium and a movable support (14) capable of receiving a cell culture made up of algae cells, which movable support is immersed at least partially in the aqueous medium and has at least a first portion and a second portion, characterised in that the movable support is arranged in the basin such that the first portion is exposed directly to a main light source (18) and forms an exposure section (24), and the second portion is not exposed directly to the main light source (18) and forms an inhibition section (26), the device (10) further comprising a secondary light source (28) designed to emit actinic light in the direction of the inhibition section (26) so as to inhibit the pigment synthesis of at least some of the algae cells. The invention also concerns a method for producing microalagae.

The present invention relates to a device and a method for producing microalgae.

Microalgae are unicellular, phototrophic, prokaryotic and eukaryotic microorganisms. Microalgae are capable of drawing their energy from light, by photosynthesis.

Prokaryotic microalgae include cyanobacteria (sometimes called “blue-green algae”). Eukaryotic microalgae are diversified and represented by a multitude of classes, which include Chlorophyceae, diatoms, Chrysophyceae, Coccolithophyceae, Euglenophyceae and Rhodopyceae.

It is currently estimated that there are more than a million species of microalgae, of which several tens of thousands of species are referenced. Microalgae are ubiquitous and are found in freshwater as well as in brackish and marine water. The size of a microalgae cell is generally between 1 μm and 100 μm.

The microalgae production industry is currently growing rapidly. Microalgae are capable of synthesising products of economic and ecological interest. These products include, in particular proteins, antioxidants, pigments, long-chain polyunsaturated fatty acids DHA (docosahexaenoic acid) and EPA (eicosapentaenoic acid).

There are also applications for microalgae in several technological fields and in particular in the cosmetics industry, pharmaceutical industry, aquaculture and the food or food supplement industry.

Microalgae are also used in bioenergy production. They have an ability to capture light energy in order to fix and metabolise inorganic carbon from carbon dioxide (CO₂) in energetic molecules. Microalgae thus have significant purifying capabilities. Moreover, the coupling of microalgae with CO₂ and the fact that microalgae are often rich in sugars or in oils have the consequence that microalgae are of great interest in the production of biofuels.

Culture systems based on growth in light are used for the production of microalgae. Hence, microalgae can be cultivated using natural light (sunlight) or artificial light.

There are open culture basin culture systems (also called “raceway” basins) and closed photobioreactor culture systems. In general, the open culture systems use sunlight, whereas the closed culture systems use artificial light.

However, the known systems have limited performance. The biomass production yield is unsatisfactory.

The present invention improves the situation.

To this effect, the invention introduces a device for producing microalgae, including a basin containing an aqueous medium and a movable support capable of receiving a cell culture made up of algae cells, said movable support is immersed at least partially in the aqueous medium and has at least two portions, characterised in that the movable support is arranged in the basin such that the first portion is directly exposed to a main light source and forms an exposure section, and the second portion is not directly exposed to the main light source and forms an inhibition section, the device further comprising a secondary light source designed to emit actinic light in the direction of the inhibition section so as to inhibit the pigment synthesis of at least some of said algae cells.

The object of the variants and preferred embodiments is this device, wherein:
The secondary light source is designed to emit light of a luminous intensity less than or equal to 30% of the average luminous intensity received by the exposure section. This light energy is optimum for good production.
The secondary light source is designed to emit light of a luminous intensity less than or equal to 300 μmol/m²/s. This light energy allows a good production while maintaining low energy expenditure and costs.
The secondary light source is designed to emit light of a luminous intensity between 5 μmol/m²/s and 300 μmol/m²/s, preferably between 30 μmol/m²/s and 120 μmol/m²/s, and more preferably approximately 50 μmol/m²/s. These energy ranges allow good production while also reducing the energy expenditure and costs.
The main light source is chosen from filtered or unfiltered sunlight and an artificial source having a wavelength between approximately 400 nm and approximately 800 nm. This light is optimum for photosynthesis. The main light source is preferably an artificial source having a wavelength between approximately 400 nm and approximately 800 nm, with a luminous intensity greater than or equal to 400 μmol/m²/s.
The secondary light source is chosen from light-emitting diodes and optical fibres. This type of source has a large flexibility for installation in the basin.
The secondary light source emits light with a wavelength between 400 nm and 550 nm. This enables, in particular, a biomass production with high yield of algae cells chosen from the genus Tetraselmis, the genus Chlorella and the genus Emiliania, preferably the species Emiliania huxleyi. In practice, LEDs are used, for which approximately 90% of the photons have a wavelength between 400 nm and 550 nm.
The secondary light source emits light with a wavelength between 590 nm and 750 nm. This enables, in particular, a biomass production with high yield of algae cells chosen from the genus Dunaliella, preferably the species Dunaliella salina, the genus Synechococcus and the genus Euglena.

Another object of the invention is a method for producing microalgae, comprising the successive exposure of a cell culture made up of algae cells to phases of direct exposure to an incident main light and phases in the shade of said incident main light, characterised in that the cell culture is also exposed to actinic light during at least some of the phases in the shade of said incident main light so as to inhibit pigment synthesis of at least some of said algae cells. This method renders at least some of the algae cells transparent.

In an embodiment, the actinic light is light with a wavelength between 400 nm and 550 nm. This can act on the pigmentation of some species of algae cells and, in particular, on microalgae chosen from the genus Tetraselmis, the genus Chlorella and the genus Emiliania, preferably the species Emiliania huxleyi.

In another embodiment, the actinic light is light with a wavelength between 590 nm and 750 nm. This can act on the pigmentation of some species of algae cells and, in particular, on microalgae chosen from the genus Dunaliella, preferably the species Dunaliella salina, the genus Synechococcus and the genus Euglena.

Preferably, the luminous intensity of the actinic light is less than or equal to 30% of the average luminous intensity received by the cell culture during the exposure phases. This reduces the energy expenditure as well as the costs of the production process.

Other advantages and features of the invention will emerge from reading the description detailed below together with the appended drawings, in which:

FIG. 1 shows a diagram of the chlorophyll content a of Emiliania huxleyi algae cells exposed to light of different colours;

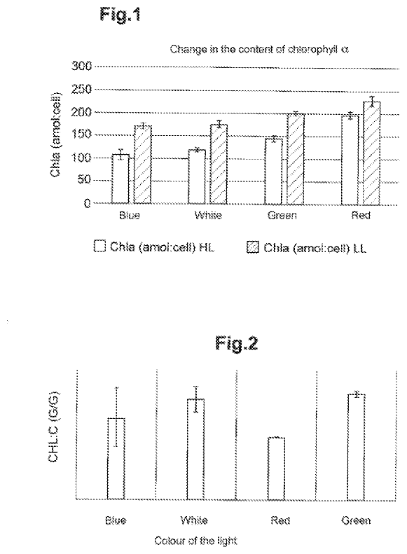

FIG. 2 shows a diagram of the chlorophyll content a per unit of cellular carbon of Dunaliella salina exposed to light of different colours;

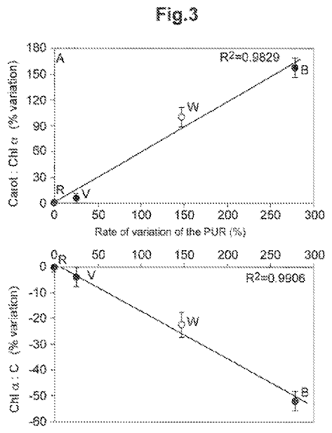

FIG. 3 shows a graph of pigment ratios as a function of the rate of variation of PUR (%) in Dunaliella salina exposed to light of different colours;

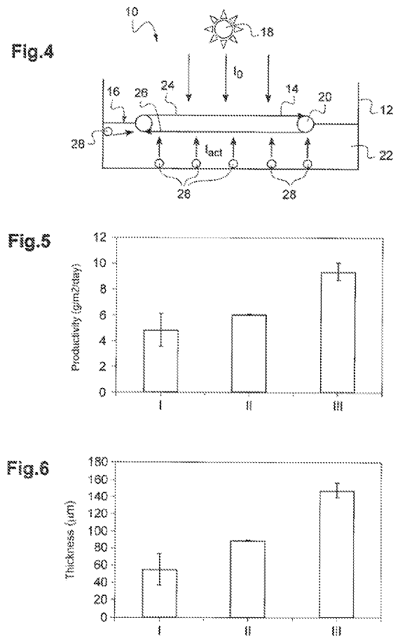

FIG. 4 shows a device according to the invention;

FIG. 5 shows a diagram of biomass productivity;

FIG. 6 shows a diagram of biofilm thickness;

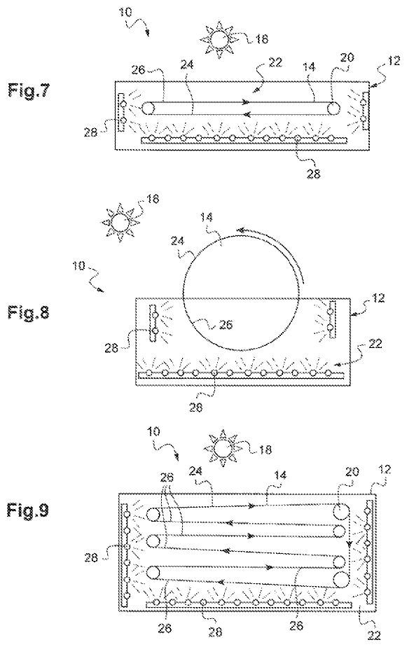

FIGS. 7, 8 and 9 show embodiments of the device of the invention; and

FIG. 10 shows a comparative diagram of biomass productivity.

The drawings and the description below essentially contain elements of a certain nature. They form an integral part of the description and can therefore not only serve as a means to better understand the present invention, but also contribute to its definition where appropriate.

All the culture systems generally include a tank or a basin filled with a culture medium. Conventionally, this is an aqueous medium. The microalgae are either dispersed in the culture medium or fixed on a support which is at least partially submerged in the culture medium. Microalgae can be described as a cell culture made up of algae cells.

Known, in particular, among the systems which include a support, is the system described in WO2015007724, filed under PCT/EP2014/065126 by the applicant on 15 Jul. 2014 and benefiting from French priority (FR) no. 13 56955 of 15 Jul. 2013. The microalgae grow on a closed loop movable support, circulating on rollers that are partially or totally immersed in the aqueous medium. This system provides a succession of phases of microalgae in sunlight and shade, while limiting the time of exposure to sunlight. Hence, the system is designed such that microalgae remain more in the shade than in sunlight. More particularly, WO2015007724 discloses a method in which the total duration of the phases in the shade is greater than 50% of the total duration of the phases of exposure to sunlight.

The applicant has discovered a means for modifying the system in a surprising manner and thus increasing the biomass production yield.

To do this, the applicant has applied the principle according to which obtaining a good productivity is linked to the illumination profile of the light to which the microalgae are subjected. Indeed, microalgae are photosynthetic species. Microalgae cells need light in order to proliferate. In general, light at a wavelength that is strongly absorbed by the microalgae is necessary in order to obtain a high growth rate. In practice, this involves sunlight or light containing at least wavelengths in the blue and red region.

The publications “Light requirements in microalgal photobioreactors: an overview of biophotonic aspects—Carvalho et al., Appl Microbiology and Biotechnology, 2011, vol. 89, no. 5: 1275-1288” and “Light emitting diodes (LEDs) applied to microalgal production—Schulze et al., Trends in Biotechnology, 2014, vol. 32, no. 8: 422-430” describe the use of light in microalgae culture systems.

Obviously, for good microalgae production under phototrophic conditions, it is necessary to provide, in addition to light, nutriments such as nitrogen, phosphorus, sulphur or silica for the diatoms in particular, trace elements and vitamins. In the presence of the necessary nutriments in the culture medium, microalgae can then engage in photosynthesis, which consists essentially of converting light energy by metabolising CO₂ and thus producing oxygen and algal biomass (organic material of microalgae).

In a production system of the type comprising a support as described in WO2015007724, the microalgae form a cluster of cells regularly distributed over the support. For example, on a belt or disc support (Algaedisk); cf. Blanken, W., Janssen, M., Cuaresma, M., Libor, Z., Bhaiji, T., & Wijffels, R. H. (2014), Biofilm growth of Chlorella sorokiniana in a rotating biological contactor based photobioreactor—Biotechnology and bioengineering, 111(12), 2436-2445. The microalgae form a biofilm on the surface of the support. The thickness of the biofilm increases with the growth of the cell culture, in other words more particularly with the cell division of the algae cells.

Pictographically, the biofilm can be seen as a plurality of superimposed cellular layers. The layers are embedded in a complex polymer structure. In a simplified manner, it can be considered that the first layer of cells is formed by the algae cells on the support and in direct contact with it. The microalgae cells constituting this first layer multiply by cell division and then form a second layer of algae cells above the first layer. The second layer divides in turn and thus generates a third layer, above the second layer, and so on. Gradually, through the cell divisions, the thickness of the biofilm increases.

However, when the biofilm reaches a certain thickness, a phenomenon called the “self-shading phenomenon” sets in. Self-shading has a negative influence on the growth of the cell culture. It is characterised by the fact that the cells at the surface, in other words the upper layers, shade the cells situated in the lower layers with respect to the light source. The cells of the lower layers do not then receive sufficient light in order to proliferate. The productivity reduces drastically.

Subsequently, harvesting of the biomass in the support systems must take place as soon as the biofilm has reached a certain thickness, preferably between 100 and 200 μm. This increases the number of steps and the costs in the production process.

The production systems with support have a main light source for providing the light necessary for photosynthesis. This source may be, for example, the sun or a lamp generating artificial light. The microalgae cells close to the main source are exposed to an excess of photons compared to the quantity of photons necessary for producing photosynthesis. This photon excess leads to a reduction in photosynthesis efficiency. It involves dissipation of energy known as non-photochemical quenching. The phenomenon of non-photochemical quenching is described, in particular, in the publication “Non-Photochemical Quenching. A Response to Excess Light Energy—Müller et al., Plant Physiol, 2001, vol. 125, no. 4: 1558-1566”. An excess of photons can also cause a degradation of the photosynthesis apparatus, called photoinhibition.

In systems with a support, the cells in the upper layer or layers of the biofilm absorb the majority of the photons coming from the main light source. A large part of the photons of the incident light then fail to penetrate deep into the biofilm in order to supply light to the cells of the lower layers. As a consequence, a light gradient is developed having a harmful effect on biomass production. Indeed, the cells of the lower layers are more or less in the dark, due to the fact that the light does not penetrate deep into the biofilm. Carbon fixation by photosynthesis does not then compensate for the energy losses due to cellular respiration, which is based on the breakdown of sugars which have been synthesised during photosynthesis.

To summarise, microalgae production systems which include a support have a problem of overexposure to light of the cells at the surface the biofilm (close to the main light source), and a problem of underexposure of cells located deep in the biofilm.

The applicant has developed a microalgae production device which solves this problem. To do this, the applicant has studied the absorption by microalgae of light at various wavelengths. It has been observed, not unsurprisingly, that some of this light has an actinic effect on the algae cells, in other words an effect which acts on the metabolic chemistry of the cells.

More particularly, certain wavelengths of light act on the pigmentation of the microalgae. Thus, by increasing the luminous intensity in one wavelength band, a depigmentation has been observed for several strains of microalgae. This results in a loss of colour of the algae cells which has the consequence that the cells become transparent.

“Transparent cells” shall be understood as cells having lost at least 20% of the pigments belonging to the group of chlorophylls or to the group of carotenoids.

FIG. 1 shows a diagram of the content (attomoles per cell: 10⁻¹⁸ moles/cell) of chlorophyll α of algae cells of the class Prymnesiophyceae (species Emiliania huxleyi) exposed to light with wavelengths corresponding to blue light, white light, green light and red light Garrido, J. L., Brunet, C., & Rodrfguez, F. (2016), Pigment variations in Emiliania huxleyi {CCMP370} as a response to changes in light intensity or quality, Environmental microbiology, 18(12), 4412-4425.

The exposure of Emiliania huxleyi cells has been carried out for each light at two different intensities:
one high (HL) at 426±60 μmol·m⁻² s⁻¹;
the other low (LL) at 16±2 μmol·m⁻² s⁻¹.

The wavelength of the blue light is 455 nm, of the red light 617 nm and of the green light 537 nm. The luminous intensity was between 250 and 450 μmol/m²/s under continuous light.

The exposure to blue light reduced the content of chlorophyll cc pigments of the algae cells from the species Emiliania huxleyi.

FIG. 2 shows a diagram of the chlorophyl α content (grams of chlorophyll α/gram of carbon) of algae cells of the class Chlorophyceae (species Dunaliella salina) exposed to light with wavelengths corresponding to blue light, white light, red light and green light for experiments carried out with constant absorbed light (PUR) Combe C., Effets quantitatifs et qualitatifs de la lumiere sur la croissance des microalgues en culture dense et sur leur production de molecules d'intérêt: vers l'optimisation des procédés de production de microalgues [Quantitative and qualitative effects of light on the growth of microalgae in dense culture and on their production of molecules of interest: towards the optimisation of microalgae production methods], doctoral thesis, Université Pierre et Marie Curie, 2016.

The wavelength of the blue light is 455 nm, of the red light 617 nm and of the green light 537 nm. The luminous intensity was between 250 and 450 μmol/m²/s under continuous light.

The exposure to red or blue light reduced the content of chlorophyll a pigments of the algae cells from the species Dunaliella salina. FIG. 3 shows the percentage increase of the ratios carotene/chlorophyll α (A) and chlorophyll α/carbon (B) as a function of the rate of variation of PUR (%) in Dunaliella salina cultivated under light with wavelengths corresponding to blue light (denoted B), white light (denoted W), green light (denoted V) and red light (denoted R).

The PUR (photosynthetic usable radiation) is calculated from the solar spectrum PARᵢₙ(λ) and the absorption spectrum characterising the microalgae Aₙ(λ).

PUR=∫₄₀₀⁷⁰⁰ PARᵢₙ(λ)Aₙ(λ)dλ

The exposure to red and blue light reduced the pigment content of the algae cells from the species Dunaliella salina.

In this case, the exposure to the red light substantially reduced the content of chlorophyll pigments of the algae cells of the species Dunaliella salina.

The addition of blue and/or red actinic light modified the pigment content of the cells with a more marked effect for the red light.

The applicant has formulated the postulate according to which a biofilm made up at least partially of algae cells with reduced pigmentation (or “transparent” according to the above definition) no longer, or almost no longer, exhibits the disadvantages of self-shading. This comes from the fact that the algae cells with reduced pigmentation allow at least some of the photons coming from the incident main light to pass. Each layer of cells, including the lower layers of the biofilm, then receive light coming from the main light source.

In the biofilm-type cultures described above, subsequently the depigmentation occurs mainly in the upper layer or layers, in other words the layer or layers of cells closest to the actinic light.

Depending on the absorption wavelength of the phytochromes of a given microalgae species, and thus on the “strong light sensation” of the cell, the blue or red light will be chosen for depigmenting the cells These phytochromes vary according to the species.

In order to systematically depigment the microalgae cell culture, the applicant uses phases referred to as “in the shade” as described in WO2015007724. In other words, the phases during which the cells are not directly exposed to the main light source. More particularly, it involves the phases during which the cells are located in zones in which the light received by the cells is of a luminous intensity less than or equal to 50% of the overall luminous intensity coming from the main light source. This applies to the overall photon flux, whatever the nature of the main light (thus independently of the incident spectrum).

However, contrary to all the embodiments of WO2015007724, in the present invention, the time during which the cells remain in the shade is not necessarily greater than the time during which the cells are exposed to the main light. In the embodiments of the present invention, the time of the phases with the cells in the shade can be equal to the time of the phases of exposure of the cells to the main light. In other embodiments, the time of the phases with the cells in the shade can be less than the time of the phases of exposure to the main light.

Hence, the device of the invention includes a movable support which has at least two portions. The movable support is arranged in a basin comprising a culture medium. Further, the support is also arranged so that the first of the two portions is directly exposed to a main light source and forms an exposure section, and the second of the two portions is not directly exposed to the main light source.

“Portion directly exposed to the main light source” shall be understood as a portion exposed to the photons emitted by the main light source. This portion is generally facing the main light source and receives the light necessary for photosynthesis. In other words, during the exposure section, the microalgae capture the light energy necessary for performing photosynthesis.

“A portion which is not directly exposed to the main light source” shall be understood as a portion at least partially in the shade for photons coming from the main light source. The cells passing through this section therefore do not receive, or receive very few, photons coming directly from the main light source

In an embodiment, over this portion in the shade, the luminous intensity coming from the main light source is less than or equal to 50% of the overall luminous intensity coming from this source. Over this portion in the shade, the luminous intensity coming from the main light source is preferably close to 0 μmol/m²/s or zero.

However, the device is designed such that the second portion is exposed to light capable of inducing a depigmentation of the algae cells This involves an actinic light which acts on the metabolic chemistry (or regulating pathways) so as to inhibit pigment production. The second portion of the movable support thus forms an inhibition section.

The inhibition of pigment production can be partial or total. A partial inhibition is manifest as a regulation reducing the synthesis of pigments; for example by blocking, reducing or slowing the transcription of genes which are responsible for the synthesis of pigments. The quantity of pigments produced in the cells is therefore reduced.

In order to expose the cells to this actinic light, the device includes a light source other than the main light source. This light source is described here as a secondary light source. The secondary light source is designed to emit actinic light in the direction of the inhibition section so as to inhibit pigment synthesis and render at least some of the algae cells transparent.

FIG. 4 shows a device 10 according to the invention. The device includes a basin 12 and a support 14 circulating in the basin 12 and on which the microalgae cells grow forming a biofilm.

The basin 12 contains an aqueous culture medium 22. The basin 12 is open in its upper part, so that the surface 16 of the aqueous medium 22 is exposed to the main light source 18 (in this case the sun). The main light source 18 emits light with a luminous intensity I₀ of approximately 400 to 2000 μmol/m²/s, depending on the meteorological conditions. The surface 16 extends over the entire open part of the basin 12.

In the present embodiment, the basin 12 is a masonry tank. In other embodiments, the basin can be a natural expanse of water, such as a lake, pond or marine bay, in particular. The basin 12 can also be a bioreactor tank.

The device further includes a movable support 14. The movable support 14 is formed of a closed-loop belt. It is guided by a set of turning and guiding rollers 20. Here, there are two rollers, respectively arranged close to the edges of the basin 12. At least one of the rollers is motorised in order to ensure the driving of the support 14.

The movable support 14 is formed by a belt having a mechanical strength. The surface of the support 14, on which the algae cells of the first layer of the biofilm will be fixed, is preferably a hydrophobic and rough support having cavities and/or micro-cavities. The support 14 has sufficient flexibility to allow passage over the rollers. It is also resistant to light, in particular ultraviolet rays. The material of the support 14 is chosen so that its eventual deterioration does not affect the metabolism of the cells.

In the example described here, the movable support comprises two portions: a first portion 24 and a second portion 26. Alternatively, the support can comprise a plurality of portions.

The rollers 20 are mounted so that their respective main axes are substantially parallel to the surface 16 of the aqueous medium 22. In this way, the first portion of the movable support 14 circulates so as to form a generally horizontal plane between the first roller 20 and the second roller at the opposite end. The first portion is arranged above the surface 16 of the aqueous medium. In the present embodiment, the first portion circulates in the atmospheric air (or, where appropriate, in controlled air when the device 10 is placed in a greenhouse). In other embodiments, the first portion is submerged in the aqueous medium 22, for example at several centimetres below the surface 16.

The movable support is subject to the turning of each roller so that the second portion 26 of the movable support 14 also circulates along a generally horizontal plane between the first roller and the second roller at the opposite end. The second portion is arranged below the surface 16 of the aqueous medium. The second portion is therefore submerged in the aqueous medium 22.

The first portion 24 is directly exposed to the light coming from the main light source 18. The second portion 26 is not directly exposed to the light coming from the main light source 18. Hence, the first portion 24 forms a section exposed to the main light source 18 and the second portion 26 forms a section in the shade of the main light source 18.

The device comprises one or more secondary light sources 28. Each secondary light source 28 is designed to emit actinic light in the direction of the section which is in the shade of the main light source 18. In this way, the cells located on the movable support 14 and passing through the section in the shade of the main source are exposed to the actinic light coming from the secondary light source or sources 28. The actinic light acts on the metabolism of the cells and inhibits pigment synthesis. At least some of the algae cells become at least partially transparent. The section in the shade of the main light source 18 is therefore described as an inhibition section. The secondary light source or sources 28 can be, in particular, light-emitting diodes or optical fibres.

In the embodiments having a plurality of portions, at least one of the portions is directly exposed to the light coming from the main light source and at least one of the portions is exposed to the actinic light coming from the secondary light source.

In the embodiment of FIG. 4, the secondary light source 28 is designed to emit light of a luminous intensity less than or equal to 30% of the average luminous intensity received by the exposure section or sections. When the main light source is sunlight and emits a luminous intensity I₀ of 2000 μmol/m²/s, the secondary light source 28 emits light of a luminous intensity I_{act} less than or equal to 600 μmol/m²/s. The secondary light source 28 is preferably designed to emit light of a luminous intensity I_{act} less than or equal to 300 μmol/m²/s. This strongly reduces the energy expenditure.

In another embodiment comprising a basin 12 of the bioreactor tank type, in order to minimise the energy expenditure, the secondary light source 28 is designed to emit light of a luminous intensity I_{act} between 5 and 300 μmol/m²/s and preferably between 30 μmol/m²/s and 120 μmol/m²/s, and preferably approximately 50 μmol/m²/s. In this embodiment, the main light source 18 is preferably a natural source having a wavelength between approximately 400 nm and approximately 800 nm with a luminous intensity greater than 400 μmol/m²/s. In this way, the energy expenditure is further reduced. This embodiment is particularly suitable for the biomass production of algae cells chosen from the genus Tetraselmis, the genus Chlorella and the genus Emiliania, preferably the species Emiliania huxleyi.

In yet another embodiment, the device 10 has a secondary light source which emits light with a wavelength between 590 nm and 750 nm. This embodiment is particularly suitable for the biomass production of algae cells chosen from the genus Dunaliella, preferably the species Dunaliella salina, the genus Synechococcus and the genus Euglena.

### EXEMPLARY EMBODIMENTS

### Example 1

The movable support 14 of a device 10 such as described above is inoculated with a cell culture made up of algae cells of the genus Tetraselmis. The speed of rotation of the rollers 20 is between 0.01 and 0.9 m/s. There are preferably three times more zones which are not directly exposed to the incident light as exposed zones. The main light source 18 is an artificial light with a luminous intensity of 400 μmol/m²/s.

In a first experiment (I), the secondary light source 28 is inactive, in other words switched off. In a second experiment (II), the secondary light source 28 emits light with a wavelength between 400 nm and 550 nm at the same location as the main light source (exposure section). Further, in a third experiment (III), the secondary light source 28 emits light with a wavelength between 400 nm and 550 nm at the inhibition section.

The temperature is constant at approximately 22° C.±1° C.

FIG. 5 shows the productivity diagram for each experiment. The fact of subjecting the algae cells to a blue actinic light over the sections in the shade of the main light source has a positive effect on the productivity. The productivity in experiment III is almost doubled with respect to experiment I.

FIG. 6 shows a diagram of the thicknesses of the biofilms for the three experiments I, II and III. The biofilm obtained in experiment III is thicker than those obtained in experiments I and II. The biomass production is increased while subjecting the algae cells to a blue actinic light over sections in the shade of the main light source.

### Example 2

Algae cells of the genus Chlorella were cultivated under the same conditions. The above results on productivity were confirmed.

### Example 3

In another embodiment, the movable support 14 of a device 10 is inoculated with a cell culture made up of algae cells of the genus Dunaliella. The experimental conditions are analogous to example 1, with the difference that the secondary light source 28 emits light with a wavelength between 590 nm and 750 nm.

The fact of subjecting the algae cells to a red actinic light over the sections in the shade of the main light source has a positive effect on the productivity. In this case, the productivity is drastically increased.

The examples demonstrate the increase in biomass yield when the cells are exposed successively to a white light coming from a main light source and an actinic light coming from a secondary light source.

The fact that the photons can penetrate to the base of the biofilm has the consequence that the self-shading phenomenon is no longer, or almost no longer, manifest. The thickness of the biofilm can thus increase without it being necessary to harvest the algae cells regularly. The steps of the production process, and hence the costs, are reduced.

FIGS. 7 to 9 show other embodiments of the device 10 of the invention.

The movable support 14 of FIG. 7 is entirely submerged in the aqueous medium. The first portion is therefore arranged in the aqueous medium below the surface 16.

FIG. 8 shows a disc-type movable support 14. This type of support is sometimes called an Algaedisk. A first portion 24 of the movable support 14 emerges from the aqueous medium 22, while a second portion 26 is submerged in the aqueous medium 22. In this way, the first portion 24 is directly exposed to a main light source 18 and forms an exposure section, and the second portion 26 is not directly exposed to the main light source and forms an inhibition section by virtue of its exposure to the actinic light emitted by a secondary light source arranged at the bottom of the basin 12. The disc turns on itself in order to alternate the phases of exposure to the light from the main light source and the phases of exposure to the actinic light coming from the secondary light source.

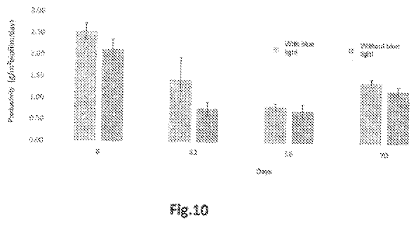

FIG. 9 shows an installation described in detail in WO2015007724. Beyond what is described in that document, the installation also includes an actinic light source 28 arranged at the bottom of the basin 12 and on its walls.

The invention can also be defined as a device for producing microalgae, including a basin containing an aqueous medium and a movable support capable of receiving a cell culture made up of algae cells, said movable support is immersed at least partially in the aqueous medium and has at least two portions, characterised in that the movable support is arranged in the basin such that the first of the two portions is exposed to sunlight or an artificial white light coming from outside the basin and forms an exposure section, and the second of the two portions is not directly exposed to said sunlight or said artificial white light and forms an inhibition section, the device further comprising an actinic light source designed to emit actinic light in the direction of the inhibition section so as to inhibit pigment synthesis and render at least some of the algae cells transparent.

The invention can again be defined as a device for producing microalgae, including a basin containing an aqueous medium and a movable support capable of receiving a cell culture made up of algae cells, said movable support is immersed at least partially in the aqueous medium and has at least a first portion and a second portion, characterised in that the movable support is arranged in the basin such that the first portion is directly exposed to a main light source and forms an exposure section, and the second portion is not directly exposed to the main light source and forms an inhibition section, the device further comprising a secondary light source designed to emit actinic light in the direction of the inhibition section so as to inhibit pigment synthesis of the cells of the cell culture directly exposed to said actinic light.

As described above, in the present description and according to the invention, actinic light is a non-photosynthetic light. In other words, actinic light according to the invention is light which is not capable of triggering the photosynthesis process in all of a microalgae cell culture. The actinic light alone does not trigger cellular growth and/or the production of microalgae biomass. More particularly here, actinic light is light for which the average intensity is less than or equal to the compensation intensity of the photosynthesis.

Yet more particularly, within the meaning of the present invention, actinic light is light triggering the inhibition of pigment synthesis. It should be noted however that this actinic light can, in rare cases, trigger the process of photosynthesis in isolated cells of a microalgae culture. This isolated photosynthesis phenomenon does not however have an effect on the increase in biomass or cellular growth in general.

In a general manner, the device is designed so that the average light (or average illumination) is substantially at the optimum level for photosynthesis, so that the actinic light is substantially below the compensation threshold. The term “average light” shall be understood as the average light received by the cells, or again the average light received per unit of biofilm. A person skilled in the art knows to identify the optimum for photosynthesis and the compensation threshold More details are given in the publications “Modelling of photosynthesis and respiration rate for Isochrysis galbana (T-Iso) and its influence on the production of this strain, Ippoliti et al., Biosource Technology 203 (2016) 71-79” and “Effects of organic carbon sources on growth, photosynthesis, and respiration of Phaeodactylum tricornutum, Xiaojuan Liu et al., J. Appl. Phycol. (2009) 21:239-246”, to which the reader is invited to refer.

In a particular embodiment, the applicant has also studied the parameterisation of the actinic light of the invention with a view to obtaining a satisfactory inhibition, or even a total inhibition, of the pigment synthesis of microalgae.

For this, the applicant started from an embodiment close to that described above.

More particularly, it involves a method in which the device includes a movable support having a first portion and a second portion. The first portion forms the exposure section on which the microalgae cells are directly exposed to the main light, in other words the photosynthetic light capable of triggering photosynthesis in algae cells. By contrast, on the second portion of the movable support the microalgae cells are not directly exposed to the main light. More precisely, on the second portion the algae cells are substantially in darkness.

Over all of the movable support, it is possible to define a surface S (m²) and an intensity I (μmol/m²/s) of light triggering photosynthesis. Logically, this surface S corresponds almost entirely to the surface of the first portion of the movable support exposed to the main light. In practice, it is not however impossible that some photons are reflected on the edges of the device and reach the isolated cells on the second portion which is in the shade of said main light. It is therefore possible, although improbable, that in some isolated cells of the second portion the photosynthesis process is triggered. However, these isolated cells and/or isolated photons are generally negligible.

It is therefore possible to measure and/or define a total light received by the microalgae and/or emitted by the device of the invention. More globally, it is possible to measure and/or define the total light in the system of the invention.

On this basis, the flux of photons of photosynthetic light (Q_{I}) supplied to the movable support is defined as follows:

Q_{I}=I×S

In parallel, the applicant is interested in the intensity of blue light I_{b}* on a surface S_{b}*. The blue light I_{b}* has a wavelength between 400 nm and 800 nm, preferably of approximately 460 nm+/−50 nm. The surface S_{b}* is a surface in the shade of the main light source. In other words, surface S_{b}* is not directly exposed to the main light. This surface is therefore defined on the second portion, as described above. It involves a sub-portion of the second portion. In this embodiment, the surface S_{b}* forms the inhibition section.

The flux of photons of non-photosynthetic light (Q_{b}) applied on the surface S_{b}* by the blue light I_{b}* is defined as follows:

Q_{b}=I_{b}*×S_{b}*

When the total surface of the movable support S_{T} is taken into account, the average flux of photons or average illumination (Q_{b-AVERAGE}) supplied on the surface S_{b}* by a non-photosynthetic blue light I_{b}* is defined as follows:

Q_{b-AVERAGE}=I_{b}*×S_{b}*/S_{T}

Further, the mean flux of photons or mean illumination (Q_{I-AVERAGE}) supplied on the surface S_{T} by the photosynthetic light I is defined as follows:

Q_{I-AVERAGE}=I×S/S_{T}

The ratio (P) between:
the non-photosynthetic light, in other words the actinic light coming from the secondary light source, and
the photosynthetic light, in other words the light coming from the main light source and possibly from the secondary light source (isolated cells), is defined as follows:

P=Q_{b}/(Q_{b}+Q_{I})

In other words, within the meaning of the present invention, P is the ratio between the actinic light and the total light.

Thus, the applicant has produced additional embodiments.

### Example 4

In this example, the movable support consists of a moving belt that is 6 m long and 1 m wide (S_{T}=6×1 m²). The speed of rotation of the belt is 0.07 m/s.

The illumination by the photosynthetic light (by means of the main light source) takes place over a length of 2 m and over the entire width of the belt.
I=200 μmol/m²/s
S=2×1 m²
→Q_{I}=400 μmol/s
→Q_{I-AVERAGE}=66.7 μmol/s

The illumination with the actinic light (by means of the secondary light source) takes place over a length of 0.1 m and over the entire width of the belt.
I_{b}=20 μmol/m²/s
S_{b}=0.1×1 m²
→Q_{b}=2 μmol/s
→Q_{b-AVERAGE}=0.33 μmol/s

Consequently: P=1%

### Example 5

In this example, the movable support consists of a moving belt that is 80 m long and 5 m wide (S_{T}=80×5 m²). The speed of rotation of the belt is 0.2 m/s.

The illumination by the photosynthetic light (by means of the main light source) takes place over a length of 20 m and over the entire width of the belt.
I=150 μmol/m²/s
S=20×5 m²
→Q_{I}=15,000 μmol/s
→Q_{I-AVERAGE}=37.5 μmol/s

The illumination with the actinic light (by means of the secondary light source) takes place over a length of 2 m and over the entire width of the belt.
I_{b}=50 μmol/m²/s
S_{b}=2×5 m²
→Q_{b}=500 μmol/s
→Q_{b-AVERAGE}=1.25 μmol/s

Consequently: P=3.3%

### Example 6

In this example, the movable support consists of a moving belt that is 8 m long and 1 m wide (ST=8×1 m²). The speed of rotation of the belt is 0.02 m/s.

The illumination by the photosynthetic light (by means of the main light source) takes place over a length of 2 m and over the entire width of the belt.
I=150 μmol/m²/s
S=2×1 m²
→Q_{I}=300 μmol/s
→Q_{I-AVERAGE}=31.25 μmol/s

The illumination with the actinic light (by means of the secondary light source) takes place over a length of 0.5 m and over the entire width of the belt.
I_{b}=50 μmol/m²/s
S_{b}=0.5×1 m²
→Q_{b}=25 μmol/s
→Q_{b-AVERAGE}=3.25 μmol/s

Consequently: P=7.7%

Subsequently, in a particular embodiment, the device of the invention is designed so that the ratio (P) between the actinic light (non-photosynthetic) and the light coming from the main light source (photosynthetic light) is less than or equal to 8%:

P≤8%

In another preferred embodiment, the ratio P is between 3% and 7%. Further, in a particularly preferred embodiment, the ratio P is from approximately 3% to approximately 3.5%. This drastically increases the biomass production.

In these particular embodiments, the present invention can be defined as follows: Device for producing microalgae, including a basin containing an aqueous medium and a movable support capable of receiving a cell culture made up of algae cells, said movable support is immersed at least partially in the aqueous medium and has at least a first portion and a second portion, wherein the movable support is arranged in the basin such that the first portion is directly exposed to a main light source and forms an exposure section, and the second portion is not directly exposed to the main light source and forms an inhibition section, the device further comprising a secondary light source designed to emit actinic light in the direction of the inhibition section so as to inhibit the pigment synthesis of at least some of said algae cells, and wherein the ratio P between the actinic light and the total light is less than or equal to 8%, preferably between 3% and 7%, and more preferably between approximately 3% and approximately 3.5%.

Hence, in this embodiment, the method of the invention can be defined as follows:

Method for producing microalgae, comprising the successive exposure of a cell culture made up of algae cells with the phases of direct exposure to an incident main light and phases in the shade of said incident main light, characterised in that the cell culture is also exposed to actinic light during at least some of the phases in the shade of said incident main light so as to inhibit pigment synthesis and render at least some of the algae cells transparent, wherein the ratio P between the actinic light and the total light is less than or equal to 8%, preferably between 3% and 7%, and more preferably between approximately 3% and approximately 3.5%.

### Example 7

A culture of microalgae is produced with the device of the invention under the following conditions:
Main light source: culture system positioned outdoors, in a greenhouse, under actual culture conditions;
Duration of the experiment: 70 days;
Triangular conveyor of dimensions 2 m×2 m×3 m;
Secondary light source: lighting system installed at 10 cm from the biofilm;
Wavelength of the actinic light: max. 463 nm+/−70 nm;
Luminous intensity of the actinic light: <100 μmol/m²/s at 10 cm;
Illuminated surface <3% of the total surface;
Strain of microalgae: Tetraselmis suecica;
Harvesting, approximately every 15 days;

In parallel, a microalgae culture is produced without actinic light, the remainder of the conditions being the same as above.

FIG. 10 shows the results. The biomass harvest is greater when using the actinic light (blue light). The device of the invention considerably increases cellular growth and thus the biomass yield. The productivity was measured in g/m² biofilm/day over a total period of 70 days.

1. Device for producing microalgae, including a basin containing an aqueous medium and a movable support capable of receiving a cell culture made up of algae cells, said movable support is immersed at least partially in the aqueous medium and has at least a first portion and a second portion, characterised in that the movable support is arranged in the basin such that the first portion is directly exposed to a main light source and forms an exposure section, and the second portion is not directly exposed to the main light source and forms an inhibition section, the device further comprising a secondary light source designed to emit actinic light in the direction of the inhibition section so as to inhibit the pigment synthesis of at least some of said algae cells.

2. Device according to claim 1, wherein the secondary light source is designed to emit light of a luminous intensity less than or equal to 30% of the average luminous intensity received by the exposure section.

3. Device according to claim 1, wherein the secondary light source is designed to emit light of a luminous intensity less than or equal to 300 μmol/m²/s.

4. Device according to claim 1, wherein the secondary light source is designed to emit light of a luminous intensity between 5 μmol/m²/s and 300 μmol/m²/s, preferably between 30 μmol/m²/s and 120 μmol/m²/s, and more preferably approximately 50 μmol/m²/s.

5. Device according to claim 1, wherein the main light source is chosen from filtered or unfiltered sunlight and an artificial source having a wavelength between approximately 400 nm and approximately 800 nm.

6. Device according to claim 5, wherein the main light source is an artificial source having a wavelength between approximately 400 nm and approximately 800 nm with a luminous intensity greater than or equal to 400 μmol/m²/s.

7. Device according to claim 1, wherein the secondary light source is chosen from light-emitting diodes and optical fibres.

8. Device according to claim 1, wherein the secondary light source emits light with a wavelength between 400 nm and 550 nm.

9. Device according to claim 8, the algae cells been chosen from the genus Tetraselmis, the genus Chlorella and the genus Emiliania, preferably the species Emiliania huxleyi.

10. Device according to claim 1, wherein the secondary light source emits light with a wavelength between 590 nm and 750 nm.

11. Device according to claim 10, the algae cells been chosen from the genus Dunaliella, preferably the species Dunaliella salina, the genus Synechococcus and the genus Euglena.

12. Method for producing microalgae, comprising the successive exposure of a cell culture made up of algae cells, with phases of direct exposure to an incident main light and phases in the shade of said incident main light, characterised in that the cell culture is also exposed to actinic light during at least some of the phases in the shade of said incident main light so as to inhibit pigment synthesis and render at least some of the algae cells transparent.

13. Method according to claim 12, wherein the actinic light is light with a wavelength between 400 nm and 550 nm.

14. Method according to claim 12, wherein the actinic light is light with a wavelength between 590 nm and 750 nm.

15. Method according to claim 12, wherein the luminous intensity of the actinic light is less than or equal to 30% of the average luminous intensity received by the cell culture during the exposure phases.

16. Method according to claim 12, wherein a ratio P between the actinic light and a total light is less than or equal to 8%, preferably between 3% and 7%, and more preferably between approximately 3% and approximately 3.5%.

17. Device according to claim 1, wherein a ratio P between the actinic light and a total light is less than or equal to 8%, preferably between 3% and 7%, and more preferably between approximately 3% and approximately 3.5%.
